(19) United States
(12) Patent Application Publication
(10) Pub. No.: US 2006/0268231 A1
(43) Pub. Date: Nov. 30, 2006

(54) Illumination method and system for obtaining color images by transcleral ophthalmic illumination

(75) Inventors: Tamir Gil, Meuchad (IL); Oded Wigderson, Haifa (IL); Amit Sasson, Herzelia (IL); Dan Laor, Haifa (IL)

Correspondence Address: BROWDY AND NEIMARK, P.L.L.C.;624 NINTH STREET, NW, SUITE 300, WASHINGTON, DC, 20001-5303, US

(73) Assignee: Medibell Medicall Vision Technologies, Ltd., Haifa (IL)

(21) Appl. No.: 10/563,293

(22) PCT Filed: Jul. 2, 2004
(86) PCT No.: PCT/US04/21269

§ 371 Date: Jun. 19, 2006

Related U.S. Application Data
(60) Provisional application No. 60/484,330, filed on Jul. 3, 2003.

(51) Int. Cl. A61B 3/10

(52) U.S. Cl. 351/221

(57) ABSTRACT

An illumination system for fundus imaging apparatus employing transcleral illumination of the interior of the eye. Color images of the interior of the eye are obtained by illuminating the sclera successively with red, yellow and green light. Those images are then treated as red, green and blue images by a post processing unit, which combines them to give a color image. This is useful for observing or imaging the interior of the eye, the retina, or the choroid. The observation or the imaging of the interior of the eye, the retina, or the choroid by applying the disclosed illumination method can be performed in conjunction with any system that includes optics for that purpose.

### BACKGROUND OF THE INVENTION

The present invention relates to ophthalmoscopes, fundus cameras, slit lamps, and operation microscopes, i.e., instruments for viewing and imaging the interior of the human eye. More particularly, the invention provides an illumination method serving to provide improved illumination for diagnostic and documentation purposes of these systems, with the possibility of avoiding pupil dilation, enlarging their observable field to the whole fundus, and bypassing illumination difficulties due to opacities and scattering in the anterior chamber of the eye. The observable field is the area of the fundus beyond which the observation system is unable to reach.

Currently, most of the fundus-viewing and imaging systems illuminate the interior of the eye through the pupil of the eye by a light source that is located in the region of a camera or other imaging device and is directed into the posterior segment of the eye. Moreover, when used to obtain color images of the retina, these systems apply light sources that produce light containing blue, green, and, red wavelengths. Because the retina is illuminated through the pupil of the eye, these systems suffer from reflections of the illuminating light off the cornea, crystalline lens, and its interface with the vitreous cavity. They need typically more than half of the pupil area for illumination, and when attempting to view portions of the interior of the eye more peripheral than the macula, the effective pupil size that is available becomes smaller and light does not go through. As a result, standard fundus viewing and imaging systems depend strongly on clear ocular media and on wide pupil dilation and they are limited to a maximum of 60° field of view and cannot observe the periphery much beyond the posterior pole. They are thus limited in patients with nondilating pupils such as those with chronic glaucoma, uveitis, and diabetes mellitus, and in patients with opaque media, cataract, and pseudophakic lens.

The problems associated with illuminating the interior of the eye through the pupil can be avoided when the interior of the eye is illuminated through the sclera (transcleral illumination), as first proposed by Pomerantzeff in U.S. Pat. No. 3,954,329. This method supports wide angle fundus imaging without demanding pupil dilation and while bypassing illumination difficulties that may rise due to obstruction and scattering from opacities in the anterior eye chamber. In addition, this method enlarges the observable field to the whole fundus.

Recently a system (Panoret-1000™ of Medibell Medical Vision Technologies, Ltd.) that is based on U.S. Pat. No. 5,966,196 (Svetliza, et al.) and U.S. Pat. No. 6,309,070 (Svetliza, et al.) has applied transcleral illumination according to the method disclosed in U.S. Pat. No. 3,954,329. The advantages and applicability of transcleral illumination as realized with the Panoret-1000™ syatem have recently been discussed by Shields et al. (Rev. Ophth. 10, 2003, Arch. Ophth. 121, 2003).

Important factors that need to be taken into account upon transcleral illumination of the interior of the eye are the optical properties of the tissue that the light goes through upon entering the eye. Before reaching the eye cavity, the light crosses the conjunctiva, the sclera, the choroid, the retinal pigment epithelium and the peripheral retina. These layers act as a red filter of light in the visual range, transmitting a maximum of 50% of red light and 10% of blue light. As a result, within eye safety limits, the amount of blue light that reaches the interior of the eye compromises very much the ability to obtain color images that would be based on red (R) green (G) and blue (B) color component contributions, so-called RGB images. In fact, analysis of fundus color images that have been obtained by directing red, green, and blue, through the sclera showed that the color images contained only red and green components, while the blue color component signal detected by the camera was weak and without features.

The fact that the blue component of the illuminating light does not reach the interior of the eye implies that the eye is exposed unnecessarily to light that does not contribute to the resulting image, and that some of the retinal findings that would be visible in three-components color images are not observed. Moreover, the signal to noise ratio (SNR) that, could theoretically be reached in a similar acquisition time but with light that would have entered the interior of the eye is thus reduced.

### BRIEF SUMMARY OF THE INVENTION

Accordingly, an object of this invention is to provide a method and a system for obtaining high-spectral and high-spatial resolution color images of the interior of the eye, one approach by applying transcleral illumination. This involves overcoming a major difficulty of illuminating the interior of the eye through the sclera with light that would be strong enough to enable the acquisition of clear and high-resolution color images without compromising eye safety. Specifically, the invention provides a novel approach to retinal imaging.

Embodiments of the invention take into account the layered structure of the eye fundus and on the fact that each layer reflects a different range of light wavelengths. Roughly speaking, when illuminating the interior of the eye, visible light within the wavelength range of 400-500 nm is mainly reflected from the surface of the retina at its interface with the vitreous cavity. Visible light within the wavelength range of 500 to 600 nm is mainly reflected from the retinal layers between the nerve fiber layer (NFL) and the retinal pigment epithelium (RPE), and visible light at wavelengths larger than 600 nm are mainly reflected from the choroid. Thus, creating a reliable color RGB image of the retina is not restricted to illuminating the retina with the conventional definitions of red, green, and blue, as long as the alternative choice of illumination bands will be reflected from the same retinal layers as would the red, green, and blue, respectively.

Broadly stated, the invention involves the recognition that there can be advantages to illuminating the retina with at least one light wavelength band that is different from red, green, or blue for obtaining a reliable retinal RGB image. Apart from improving transmittance, use could be made of a particular light producing technology that has many advantages, such as lasers or LEDs, that cannot provide the standard Red, Green, and Blue wavelength bands at high enough intensities. According to the invention, such technologies could still be used for the acquisition of “RGB” color images of the retina if providing other nearby wavelength bands, or wavelengths.

Without losing generality, the invention is preferably applied to retinal imaging to systems based on transcleral illumination by shifting the lower limit of the illumination spectrum to wavelength values that are transmitted by the sclera, yet dividing the spectrum into three ranges in order to enhance both the signal/noise ratio and the spectral resolution. By way of preferred example, according to the present invention the sclera is illuminated with red, yellow, and green (RYG) light beams instead of red, green, and blue (RGB) light beams that would typically be used to compose a color image (see U.S. Pat. No. 6,309,070, Svetliza, et al.).

This preferred example of the invention takes advantage of the fact that the sclera transmits yellow light twice as much as blue and of the fact that the yellow light is much less hazardous to eye tissues. Moreover, alignment and focusing of the imaging optics as preparation for the color acquisition is done under yellow light alone, which is the longest wavelength and least hazardous light component that still images the retina and not the choroid. Color images of the interior of the eye are then obtained by taking the R, Y, and G corresponding grey-level-coded images of the fundus and converting them into red-green-blue (RGB) images by a post processing unit, which combines them to give a color image. Specifically, the red component of the color image is given by the red-illuminated image, the green component by the yellow-illuminated image, and the blue component by the green-illuminated image.

Given this invention, transcleral illumination with its aforementioned advantages will yield images with higher than heretofore spectral and spatial resolution and signal-to-noise ratio (SNR).

In accordance with a preferred embodiment of the present invention there is provided a method for integrated ophthalmic illumination comprising the steps of:
illuminating, preferably sequentially, a region within the eye with light of a plurality of different colors, one of which is yellow; and
forming images, preferably sequential, of the eye region provided by light of each of the different colors.

A more specific implementation of the invention comprises:
providing a light source producing a light beam;
separating red, yellow, and green components of said light beam;
sequentially illuminating the eye with said separated red, yellow, and green components at a rate of one component per frame;
imaging said sequentially illuminated subject;
and
processing said sequential color images such that said separated red, yellow, and green components are combined as red, green, and blue (RGB) components so as to obtain a high resolution color image.

The invention also provides an integrated ophthalmic illumination apparatus that basically comprises:
means for sequentially illuminating a region within the eye with light of a plurality of different colors, one of which is yellow; and
means for forming sequential images of the eye region provided by light of each of the different colors.

More specifically, the integrated ophthalmic illumination system may comprise:
a light source for producing light having a plurality of color components;
an optical filter unit disposed in the path of the illumination beam for selecting only light wavelengths that are required for imaging while avoiding unnecessary irradiation of the eye;
a separation unit for sequentially separating light from said optical filter unit into red, yellow, and green color components of said light beam;
an optical system disposed for directing each of the light color components sequentially into a region within the eye, so as to produce sequential gray-level-coded images;
an image capturing device disposed to obtain successive images of the region within the eye provided by each of the color components; and
a computer processor connected to form from the successive images at least one of a high resolution color image and a monochromatic image.

In the preferred embodiment of the invention, there is provided an illumination system having a lamp (including but not limited to a tungsten, metal halide or halogen lamp or any type of filament, arc, gas, laser, or semi-conductor diode lamp). In a preferred embodiment, color images are provided using a red-yellow-green-transparent (RYGT) filter wheel. The filter wheel is divided into four sections or arc sections around the periphery of the wheel. Three of the four partitioned sections are larger than the fourth and equal to one another and comprise the three optical R, Y, and G filter sections. The fourth section is a transparent, or empty, narrow section that is used for transferring the full original content of the white beam when a monochromatic or chromatic image is desired, e.g., in order to emit a fluorescence exciting light beam. Alternatively, this narrow section can be a filter that provides a fluorescence exciting beam, or, a near-infra-red (NIR) filter that allows alignment and focusing of the imaging optics without being sensed by the patient's eye and without causing pupil dilation.

In order to produce color images, the RYGT wheel rotates at a speed of one third of the frame rate of a CCD camera, producing a sequence of definite R, Y and G spectral light bursts. These light bursts illuminate the interior of the eye, enabling an image of the eye fundus to be reflected out and detected by the image capturing sensor. These R, Y, and G-illuminated images are later composed by a computer into a single colored picture.

In order to produce monochromatic images, the RYGT wheel stays in a fixed position at which the light passes through the transparent (T) section, or, alternatively through this section when it serves as a filter, producing a fluorescence exciting beam, or, a near-infra-red (NIR) filter for alignment and focusing of the imaging optics.

In order to enable alignment and focusing of the imaging optics under near-infra-red (NIR) illumination, thus reducing stimulation of the sensory retina, improving patient comfort, and avoiding pupil contraction before color image acquisition, two alternative embodiments are presented. One includes a long-pass filter to transmit near-infra-red (NIR) from the light source. The other one comprises a short-pass filter that in synchronization with a long-pass red segment of the RYG filter wheel yields a band-pass near-infra-red (NIR) illumination. Switching to acquisition a color image after performing NIR focus and alignment of the imaging optics is supported by a mechanism for fast exchange of the low-pass with the hot mirror band-pass filter.

In an alternative embodiment, similar color splitting is accomplished by means of an X-cube splitter used to divide the white light into its R, Y and G components. In yet another preferred embodiment, a series of three 45° tilted beam splitters or dichroic spectral beam splitters are used to divide the light into three channels, and then the desired wavelengths are filtered from each channel.

In yet another alternative embodiment of the patent the aforementioned lamp is replaced by an array of many smaller light sources. By way of example, laser diodes or light emitting diodes (LED) are arranged on a spherical surface with their principal light emission axis perpendicular to that surface. As a result, most of the light energy that is emitted by these diodes is concentrated at the center of the sphere, creating a small light spot that corresponds in size to the light-emitting gap in a single diode chip but has the energy that is the sum of the energies emitted from all the diodes together. Collimating optics is applied to each one of the diode sources bringing the size of the light spot at the center of the sphere down to an order of magnitude of hundreds of microns. Accordingly, entrance aperture 10 (FIG. 2) is centered at this point, efficiently transmitting the light into the optical fiber 11. The numerical aperture of the optical fiber determines the maximal angular opening of the spherical segment on which the diodes are arranged. Accordingly, the larger the radius of the sphere, the greater the number of diodes arranged on it can be. Spectral characteristics of the diode arrays are determined by the choice of diodes put in the array.

Other features and advantages of the invention will become apparent from the following drawings and description.

### BRIEF DESCRIPTION OF THE DRAWINGS

For a better understanding of the invention with regard to the embodiments thereof, reference is made to the accompanying drawings, in which like numerals designate corresponding elements or sections throughout, and in which:

FIG. 1 is a pictorial view of a first embodiment of the illumination system of the present invention;

FIG. 2 is a pictorial view of an RYGT filter wheel provided in the system of FIG. 1;

FIG. 3 is a pictorial view of a second embodiment of the illumination system of the present invention;

FIG. 4 is a pictorial view of a third embodiment of the illumination system of the present invention; and

FIG. 5 is a block diagram of one embodiment of the computerized controls for illumination systems of the present invention.

### DETAILED DESCRIPTION OF THE INVENTION

Without losing generality, there is described herein a first exemplary embodiment of the present invention which is a modification of an existing illumination system of Panoret-1000™ (Medibell Medical Vision Technologies, Ltd.), built in accordance with U.S. Pat. No. 6,309,070 (Svetliza, et al.).

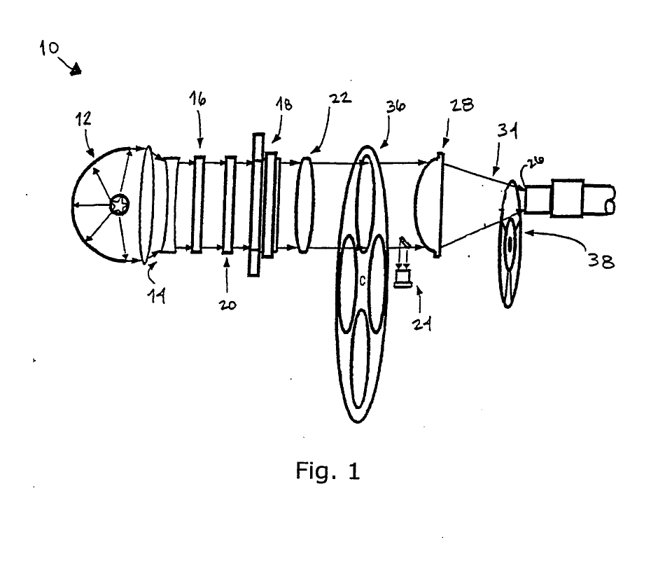

Referring to FIG. 1, there is shown an illumination system 10, in which a lamp 12 (by way of example a tungsten, halogen or metal-halide lamp or any type of arc, filament or gas lamp) produces a well-defined collimated light beam, with the aid of matching beam-expander optics 14. A hot mirror 16 is placed in the optical path close to the light source to remove ultraviolet (UV) and infrared (IR) components of the light spectral content. An electro-optical fast shutter 18 (by way of example, an LCP250 scattering liquid crystal polymer shutter, by Philips, the Netherlands) controls the amount of light in the collimated beam that traverses the shutter by changing the shutter light scattering effectiveness (i.e. its direct transmission). A neutral density filter 20 may be inserted to enable a more pronounced light power change in the traversing beam. It is used during alignment and focusing of the imaging optics, serving to avoid exposing the eye of the patient to uncomfortable strong light during this process that takes much longer than the actual recording of the image takes place, where the full illumination power is required in order to obtain a high quality image. Thus, it is quickly removed upon image recording and falls back into place afterwards. When coming to improve patient comfort even further, in an alternative embodiment, the neutral density filter may be coupled with a near-infra-red (NIR). Hence, reducing to minimum the exposure the patient to visible light during alignment and focusing.

Additional correction optics, e.g. 22, may also be placed downstream of the optical path for beam correction and shaping.

A photodiode 24 monitors the overall light intensity within the optical beam, aided by a beam splitter 25 that is introduced into the collimated beam so as to reflect a small fraction of the main beam light to photodiode 24. This mode of light measurement provides an important safety feature when used with sensitive tissue, such as tissue in the eye.

Towards the end of the light path, the collimated beam is focused onto an entrance aperture 26 of a fiber optics feeding cable using a short focusing aspheric condensing lens 28. A short focus lens is recommended in order to minimize the beam spot-size dimensions on the entrance aperture plane of the fiber optics bundle guide.

The filters of a rotary wheel 36 may be positioned in the optical path for monochromatic illumination. Rotary filter wheel 36 has several radially spaced filters mounted on a disc. Wheel 36 locks in certain positions where one of the interchangeable filters intercepts the entire beam cross section, thus isolating a certain spectral window from the fill “white” content of the beam. This enables a specified spectral band or colored illumination to illuminate the subject. The monochromatic filters of the rotary wheel may be used also as excitation filters for Fluorescein or Indocyanine Green angiography. By way of example, filter wheel 36 may be provided with narrow band-pass optical filters and a transparent (T) or empty window. When filter wheel 36 is locked in position so that the transparent or empty window intercepts the beam cross section, the full power and spectral content of the light beam can be transferred to the next station.

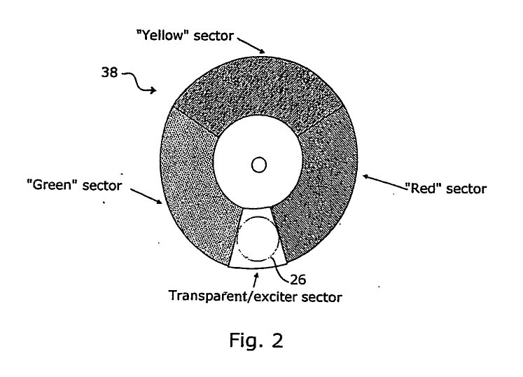

In order to enable color imaging without any loss of the high resolution available from a black and white CCD camera, a second RYGT filter wheel 38 is provided in the optical path. As shown in FIG. 2, this wheel is divided, by way of example, into four partitioned section, the R, Y and G sections being larger and equal to one another and the smaller fourth section, a T section that is used for transferring the full original content of the white beam. The dimensions of the T section, at a minimum, cover the cross-section of fiber optic cable aperture 26.

In an alternative embodiment, instead of the transparent (T) section in the RYGT wheel, there can be provided a narrow-band filter for passing a wavelength range that is appropriate for exciting a fluorescent dye for angiographic applications, e.g., a blue filter for Fluorescein Angiography, or a near-infra-red filter for Indocyanine Green (ICG) angiongraphy.

In yet another alternative embodiment, instead of the transparent (T) section in the RYGT wheel, there can be provided a near infra red (NIR) filter for passing a wavelength range higher than 700 nm to which the human eye is not sensitive, while the camera used to acquire the retinal images is sensitive. In this case, during alignment and focusing of the imaging optics, the NIR section is placed in the center of the illumination beam, having its full cross section included in it. Accordingly, during alignment and focusing, the examined patient is not disturbed by the light shined onto the retina nor the pupil contracts, while the retinal image acquired by the camera appears as a black and white image on the computer monitor. Concomitantly, when recording a color image of the retina, the filter wheel is accelerated in a controlled mode to pass each on of the R, Y, G segments at the rate of the camera frame and in synchronization with it.

In order to establish the highest achievable duty cycle for each of the three main R, Y and G colored sections, RYGT wheel 38 is preferably positioned close to a plane where the beam is narrowed to a minimum (i.e. near the focal plane of fiber optics outlet port aperture 26). With wheel 38 thus positioned, the projection of the beam cross-section is small, meaning that the T section of the wheel can have the smallest possible size while still covering aperture 26. This allows the largest duty cycle for the three remaining color filter sections, RYG. When RYGT wheel 38 rotates at a speed of one third of the frame rate of the CCD camera, a sequence of definite R, Y and G (with a short white) spectral light bursts are transferred to aperture 26 for each revolution of RYGT wheel 38. Each of these R, Y and G sequenced light bursts is fully synchronized with one of the consecutive frames of the CCD camera located in the detection channel. This produces R, Y and G illuminated images in sequence, each frame of the camera having one color. These images are later composed by the computer into a single full color picture. Thus, every three consecutive monochromatic “colored” images comprise one colored picture. The computer updates these colored pictures at the rate of the camera frame rate, each time a new “colored” frame is detected.

Referring again to FIG. 1, when color pictures are no longer required, RYGT wheel 38 is locked in a position where the T section extends across the beam cross-section, allowing the full impinging light content from lamp 12 to be passed to aperture 26. When locked in this “white” position, the light can be used for angiography or for specific monochromatic illumination purposes by introducing the appropriate filters into the optical path using filter wheel 36.

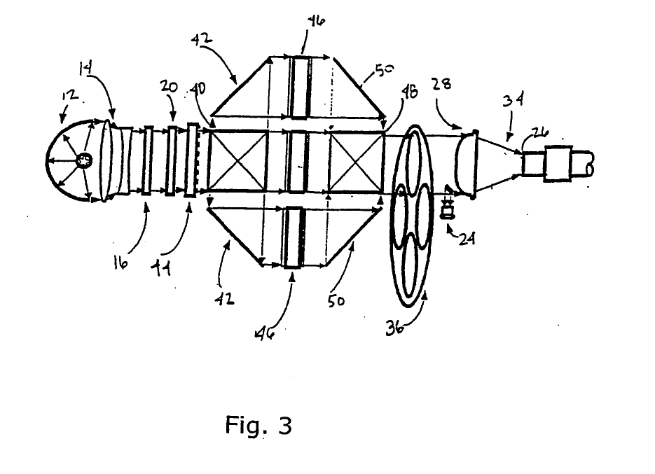

FIG. 3 shows a second embodiment of illumination system 10, having a light path similar to that of FIG. 1, in which a halogen or metal-halide lamp 12 produces a well-defined collimated light beam, with the aid of matching beam-expander optics 14. Hot mirror 16 is placed in the optical path close to the light source to remove ultraviolet (UV) and infrared (IR) components of the light spectral content. In this embodiment, the main beam is split into three “colored” channels (R, Y, G) using R-Y-G dichroic “X-cube” splitter 40 with two 45° tilted mirrors 42 that deflect the side emerging channel beams to produce three parallel beams. To overcome a possible loss of some polarized light beam components due to polarization sensitivity of X-cube splitter 40, a polarization converter prism 44 is inserted in the light path preceding X-cube splitter 40, so as to transform the impinging randomly polarized light beam into a linearly polarized one.

Three electro-optical fast shutters 46 (by way of example, LCP250 scattering liquid crystal polymer shutters, by Philips, the Netherlands) are placed in each of the three split channels to switch on the channels sequentially, each for a duration of one camera frame. Beside the act of switching, shutters 46 are also used for controlling the beam power in each of the channels in order to correctly balance the light power relationship among the three channels.

The three separated channels may be recombined into a single beam by an X-cube combiner 48, with the aid of two 45° tilted mirrors 50. When the three (RYG) shutters are operated sequentially so that each conducts light during one camera frame duration, red, yellow, and green light bursts sequentially emerge from X-cube combiner 48. Focusing lens 28 is used to focus the emerged collimated beam onto aperture 26. When colored pictures are not required, all of fast shutters 46 are kept locked in their transparent mode. The combined R, Y and G beams together constitute a white light beam that is passed to aperture 26. As in FIG. 1, the white beam illumination can be used for angiography or for specific monochromatic illumination by introducing the appropriate filter into filter wheel 36.

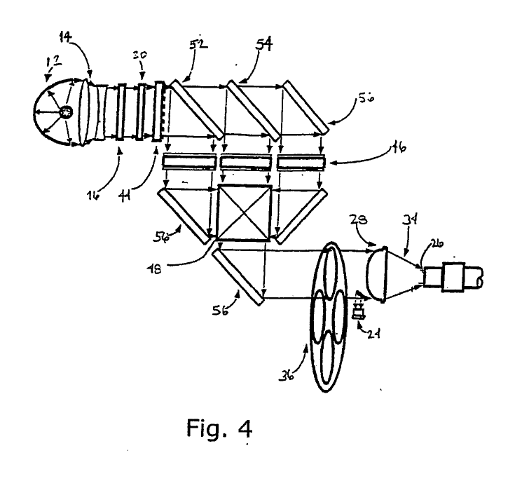

Referring now to FIG. 4, a third embodiment of illumination system 10 is shown in which the splitting of the main channel into R, Y and G sequential synchronized light bursts is accomplished using a series of three 45° tilted beam splitters: 30R/70T (30% reflecting/70% transmitting) beam splitter 52, 50R/50T beam splitter 54 and 45° tilted mirror 56, and adding an R, Y or G optical filter to each of the channels. Alternatively, a series of three 45° tilted dichroic spectral beam splitters for R, Y and G may be used (e.g. J43-454, J43-455 and J43-458 correctors marketed by Edmund Scientific, Barrington, N.J., USA).

The use of three 45° tilted beam splitters is the least efficient method of color splitting, as compared to the embodiments shown in FIGS. 1 and 3, due to the partitioning of the total beam power into three separated channels with about one third of the total power content in each channel. Therefore, the optical filters in each channel separate out only part of the spectral content of the already reduced light power in the channel. Once the color splitting has been accomplished, mirrors 44 and X-cube combiner 48 function as described with reference to FIG. 3.

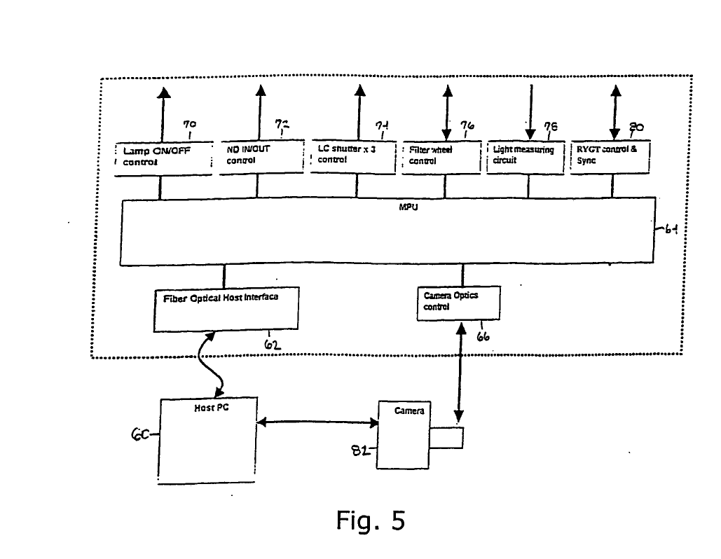

Referring now to FIG. 5, there is shown a block diagram of the computerized controls for illumination system 10, provided as a printed circuit board (PCB) designed to control and monitor the optical parts of illumination system 10 in any of the embodiments depicted in FIGS. 1, 3 or 4, and to interface with a host PC 60. These computerized controls are disclosed in U.S. Pat. No. 6,309,070, the disclosure of which is incorporated herein by reference.

In block 62, a copper-to-fiber interface between the PC 60 and the illumination system is provided as a fiber optic interface for signal conversion, with communication of up to 100 Mbit/sec, bidirectionally. In block 64, the main processing unit (MPU), which may be, for example an Altera-based type, is in charge of communication with all I/O's and host PC 60. The control algorithms are implemented here.

In block 66 there is an option for camera optics control. A circuit in block 70 controls lamp 12. This may also be used as an emergency off circuit. Neutral density filter 20 is inserted or removed by block 72 to control light passing therethrough from light source 12. In block 74, there is provided a circuit capable of controlling up to three fast shutters such as 18 or 46, for continuous control frame resolution and color weighing.

The filter wheel control is provided in block 76 and drives rotary filter wheel 36. An 8-channel 10-bit serial analog-to-digital converter (ADC) is provided in block 78 for measuring light passing through the light source and for monitoring safe light levels in the light measuring circuit. Block 80 is a circuit used to revolve color wheel 38 so it is synchronized to the camera frame integration in color mode, and to position the wheel in its transparent sector in monochromatic and angiography test modes.

Clearly, the present invention may interface with the illumination path of a slit lamp, any kind of opththalmoscope, ophthalmic camera, surgical microscope, endoscope, culposcope, laparascope, or other medical device. In this way these devices become versatile, allowing a wide range of test capability with a single optical system which includes color, monochromatic and angiography imaging ability.

If it is desired to illuminate the retina with wavelengths other than red and blue, suitable light sources or filters can be provided. For illumination through the pupil, use can be made of known optical illumination and detecting systems of that type, modified to provide the desired illumination wavelengths. Conversion of the received light to RGB components is achieved according to principles known in the art.

Having described the invention with regard to certain specific embodiments thereof, it is to be understood that the description is not meant as a limitation, since further modifications will be apparent to those skilled in the art, and it is intended to cover such modifications as fall within the scope of the appended claims.

What is claimed is:

1. A method for integrated ophthalmic illumination comprising:
illuminating a region within the eye with light of a plurality of different colors, one of which is yellow; and
forming images of the eye region provided by light of each of the different colors.

2. The method of claim 1, wherein the images are formed sequentially, and further comprising:
combining the sequential images so as to obtain a combined image.

3. The method of claim 2 wherein the different colors further include red and green and said step of forming images includes treating the images provided by red, yellow and green light as the red, green and blue image components, respectively, and the combined image is a high resolution color image.

4. The method of claim 1 wherein said step of illuminating is performed by supplying light from a lamp source composed of a plurality of smaller light sources that each provide light of a respective one of the plurality of different colors.

5. An integrated ophthalmic illumination apparatus comprising:
means for sequentially illuminating a region within the eye with light of a plurality of different colors, one of which is yellow; and
means for forming sequential images of the eye region provided by light of each of the different colors.

6. An integrated ophthalmic illumination apparatus comprising:
a light source for producing light having a plurality of color components;
an optical filter unit disposed in the path of the illumination beam for selecting only light wavelengths that are required for imaging while avoiding unnecessary irradiation of the eye;
a separation unit for sequentially separating light from said optical filter unit into red, yellow, and green color components of said light beam;
an optical system disposed for directing each of the light color components sequentially into a region within the eye, so as to produce sequential color images;
an image capturing device disposed to obtain successive images of the region within the eye provided by each of the color components; and
X a computer processor connected to form from the successive images at least one of a high resolution color image and a monochromatic image.

7. The apparatus of claim 6 wherein said light source is one of: an arc lamp; a filament lamp; a gas lamp; a laser; and a plurality of diodes.

8. The apparatus of claim 6 further comprising electronically-controlled means for controlling the intensity of the light color components directed into the region of the eye.

9. The apparatus of claim 8 wherein said electronically-controlled means comprise a fast liquid crystal shutter.

10. The apparatus of claim 9 wherein said processor controls said shutter for each image obtained by said image capturing device.

11. The apparatus of claim 6 wherein said separation unit is a filter wheel.

12. The apparatus of claim 11 wherein said imaging means operates at a frame rate and said filter wheel rotates at a speed of one-third of the frame rate of said imaging means.

13. The apparatus of claim 6 wherein said separation unit is a filter wheel having three color filters, and further comprising electronically-controlled means for controlling the intensity of the light color components directed into the region of the eye, wherein said electronically-controlled means are operative for allowing light through not more than two of said filters during image alignment and focusing of the imaging optics in order to reduce the amount of light directed onto the eye, while enhancing for focus a chosen retinal layer by choosing an appropriate illumination.

14. The apparatus of claim 13 wherein images acquired during selected illumination are displayed as grey-level, black and white, images during alignment and focusing of the imaging optics in order to improve visual contrast.

15. The apparatus of claim 6 further comprising a neutral density filter disposed in the path of light from the source or in the path of the collimated beam.

16. The apparatus of claim 6 further comprising a long-pass optical filter to transmit near-infra-red radiation from the light source.

17. The apparatus of claim 6 wherein said separation unit comprises a long-pass red filter, and further comprising a short-pass optical filter that acts together with said red filter to form a band-pass near-infra-red transmitting filter.

18. The apparatus of claim 17, further comprising a mechanism for effecting fast withdrawal of said short-pass optical filter for switching from near-infra-red imaging to color imaging.

19. The apparatus of claim 6 further wherein said optical system comprises a condensing lens for reducing the diameter of said illuminating light beam.

20. The apparatus of claim 6 wherein said image capturing device is a monochrome electronic imaging sensor.

21. The apparatus of claim 6 wherein said image capturing device is a color camera.

22. The apparatus of claim 6 wherein said separation unit comprises: an RYG dichroic X-cube splitter producing two deflected side emerging channel beams; two tilted mirrors that deflect said side emerging channel beams to provide two light beams parallel to said collimated light beam; and an X-cube combiner.

23. The apparatus of claim 6 wherein said separation unit comprises a series of tilted beam splitters.

24. The apparatus of claim 6 wherein said light source comprises a plurality of small light sources.

25. The apparatus of claim 6 wherein said separation unit is a filter wheel having three color filters, and at least two of said filters have respectively different sizes, yielding different exposure and frame times for each color in order to compensate for the selective color transmittance of the sclera.

26. The apparatus of claim 6 wherein said separation unit is a filter wheel having red, yellow, and green color filter sections substantially equal in size and a transparent section smaller than each of said color filter sections.

27. The apparatus of claim 6 wherein said separation unit is a filter wheel having three color filters, and further comprising a second filter wheel for use in at least one of monochromatic illumination and excitation with angiographic agents.

28. The apparatus of claim 27 wherein said second filter wheel further comprises a transparent section to allow the full spectral content of said light beam to pass through said first filter wheel.

29. The apparatus of claim 6 wherein said separation unit is a filter wheel comprising four filter sections, with sections for red, yellow, and green being substantially equal in size and said red, yellow, and green sections being larger than a section of wavelength range adequate for excitation of angiographic agents.

30. Claim 29 wherein the section of wavelength range adequate for the excitation of angiographic agents is a blue section that is adequate for fluorescein angiography.

31. Claim 29 wherein the section of wavelength range adequate for the excitation of angiographic agents is a near-infra-red section that is adequate for indocyanine green angiography.

32. The apparatus of claim 6 wherein said separation unit is a filter wheel comprising four filter sections, with sections for red, yellow, and green being substantially equal in size and said red, yellow, and green sections being larger than a near-infra-red section of wavelengths range adequate for imaging the retina during alignment and focusing of the imaging optics without stimulating the sensory retina, improving patient comfort, and avoiding eye pupil contraction before color image acquisition.

33. A method for integrated ophthalmic illumination comprising:
illuminating a region within the eye with light of a plurality of different colors, at least one of which is different from any one of red, green and blue; and
forming images of the eye region provided by light of each of the different colors, the images being composed of red, green and blue components, each component being derived from illumination of a respective one of the different illuminating colors.
